(19) United States
(12) Patent Application Publication
(10) Pub. No.: US 2006/0030779 A1
(43) Pub. Date: Feb. 9, 2006

(54) Intensity level control for contrast agent imaging

(75) Inventors: James E. Chomas, San Francisco, CA (US); Edward A. Gardner, San Jose, CA (US); Patrick J. Phillips, Sunnyvale, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc.

(21) Appl. No.: 10/914,850

(22) Filed: Aug. 9, 2004

(51) Int. Cl. A61B 8/14

(52) U.S. Cl. 600/458

(57) ABSTRACT

A target mechanical index, acoustic pressure, intensity or other value associated with contrast agent imaging is established. For contrast agent imaging associated with limited destruction of contrast agents, the transmit power is matched to the target intensity value as a function of position, time or combinations thereof. Given multiple foci, the transmit power is adjusted to provide a substantially uniform acoustic pressure or intensity at two or more of the foci. Where an imaging parameter is adjusted during an imaging session, the transmit power is altered to provide a similar or same target intensity value at a given location that is consistent with the value before the adjustment. By setting the transmit power as a function of a target value, more consistent contrast agent imaging may be provided despite the use of multiple foci, changes in the location of one or more foci, or changes in other imaging parameters. By automating setting the transmit power as a function of the target value, user adjustment may be avoided. A target acoustic pressure, intensity or mechanical index value may be used for high power more destructive imaging as well.

### BACKGROUND

The present invention relates to intensity level control for contrast agent imaging. In particular, more uniform pressure is provided in different contrast agent imaging configurations or environments.

Contrast agents have a non-linear response to transmit power. Various contrast agent imaging modes are responsive to non-linear response. These contrast imaging modes are typically adapted to limit or minimize destruction of contrast agents, such as being associated with lower mechanical index imaging. Imaging performance for contrast agent imaging modes is dependent on the transmitted acoustic pressure. Mechanical index is one measure of acoustic pressure.

Lowering the mechanical index and associated transmit power may reduce the signal-to-noise ratio. FIG. 1 shows the relationship between persistence or maintaining contrast agent, the signal-to-noise ratio of received signals, and the mechanical index at a focal location. The signal-to-noise ratio used in FIG. 1 is the signal in a region of contrast agents divided by the signal in a region of tissue at a depth of 50 millimeters. Persistence is measured as a total time of contrast agent signal being observed from the start of imaging. The example of FIG. 1 is measured using a 4C1 transducer at 2 megahertz on a Siemens Sequoiag ultrasound system using contrast pulse sequences contrast agent imaging. The contrast agent used was Levovist. For high mechanical index values, the persistence is low, reflecting destruction of contrast agent. For low mechanical index values at the focus, the persistence time is higher but the signal-to-noise ratio decreases. A more optimal mechanical index at the focus for contrast agent imaging with limited destruction of contrast agents causes a long persistence of contrast agent signal while maximizing the signal-to-noise ratio of the contrast agents. In the example of FIG. 1, one more optimal mechanical index at the focus is 0.29, but optimal performance based on lesser or greater persistence and relatively greater or lesser signal-to-noise ratio may be used. Different transducers, contrast agents, detection methods, frequencies or other characteristics may provide different more optimal values.

To obtain a low acoustic pressure for limiting the destruction of contrast agents, a power management model implemented by an ultrasound imaging system limits the transmit power. In one example, a mechanical index associated with the transmit powers being maximized is calculated. If the mechanical index anywhere within a scanned region exceeds a government regulation, such as a 1.9 value, the transmit power is reduced until the mechanical index is not exceeded. For imaging modes associated with destruction of contrast agent or imaging without contrast agent, the maximum allowable power is then used for transmitting acoustic energy. For contrast agent imaging with limited destruction of contrast agents, the transmit power is reduced. For example, a user adjusts a knob indicating a lesser or reduced transmit power. The maximum allowable transmit power is then reduced by an amount input by the user (% or dB). The user may increase or decrease the transmit power in a manual attempt to increase or decrease the signal-to-noise ratio and associated persistence.

Different imaging parameter values may result in different peak pressure given either a same transmit voltage or an updated transmit voltage. For example, an image is formed using two different focal depths per scan line. As shown in FIG. 1, one focal depth is provided at 45 millimeters and a second focal depth at 90 millimeters. The dashed lines corresponding to the two depths indicate a difference in both signal-to-noise ratios as well as persistence at each of the depths given a same transmit power of −16 db. The resulting mechanical index at the two different focal regions is also different, such as 0.35 for the shallow depth and 0.24 for the deeper depth. This difference in mechanical index at the two different focal points has a difference of 16 db in the signal-to-noise ratio and about 100 seconds difference in persistence. The difference in mechanical index and associated acoustic pressure result in variation within a same image. For high frequency contrast agent imaging or a long duration perfusion study, the difference may be even more significant.

The difference in mechanical index and associated variations in the image may differ as a function of the depth of the focal depths. FIG. 2 shows variation as a function of different combinations of focal depths using the system and configuration discussed above with respect to FIG. 1. The screen mechanical index corresponds to a highest mechanical index within a scan region whether at a focal position or not. The screen mechanical index corresponds to the mechanical index measured pursuant to the FDA regulations. The screen mechanical index at the focus (MIF) represents a mechanical index associated with the deepest focal point in a dual focus imaging mode. The MIF shallow and deep graphs represent a measured mechanical index at each of the shallow and deep focal points. The variation of the screen mechanical index from the mechanical index at the shallow focal point deviates for deeper depths due to the mechanical elevation focus of the transducer. The focal depth axis of the graph of FIG. 2 shows various paired combinations in millimeters of shallow and deep focal points. Since low mechanical index imaging is used, the mechanical index is unlikely to exceed the FDA regulations. Using the low MIF contrast agent imaging power settings discussed above, the transmit power at the transducer for each of the focal depths is the same. The resulting mechanical index at the various focal depths however varies over the range of possible focal positions. The mechanical index at the shallow focus maintains a relatively constant MIF, but the mechanical index at the deeper focus decreases significantly for deeper depths. For deeper depths, the difference in mechanical index at the shallow and deep focal points results in different signal-to-noise ratios and persistences. For example, a 0.12 difference in mechanical index is provided for the 45 and 90 millimeter combination of focal points. The difference in mechanical index results in a significant difference in contrast agent sensitivity and/or unnecessary contrast agent destruction.

Differences in acoustic pressure may also result due to changes as a function of time. When the user changes an imaging parameter during an imaging session, such as changing a transmit frequency, focal position, or other imaging parameter, the mechanical index may be varied. For low MI contrast agent imaging, a change is unlikely to result in exceeding MI limitations. As a result, the transmit power is not further adjusted automatically. With the different imaging parameter and the same transmit power, a different mechanical index or mechanical index at the focus may result. The difference in mechanical index may appear unpredictable to a user, reducing a consistency of contrast agent imaging performance and/or resulting in a manual adjustment of power by the user. The user either constantly adjusts the power or accepts variation in mechanical index.

### BRIEF SUMMARY

By way of introduction, the preferred embodiments described below include methods and systems for controlling an intensity level for contrast agent imaging. A target mechanical index, acoustic pressure, intensity or other value associated with contrast agent imaging is established. For contrast agent imaging associated with limited destruction of contrast agents, the transmit power is matched to the target intensity value as a function of position, time or combinations thereof. Given multiple foci, the transmit power is adjusted to provide a substantially uniform acoustic pressure or intensity at two or more of the foci. Where an imaging parameter is adjusted during an imaging session, the transmit power is altered to provide a similar or same target intensity value at a given location that is consistent with the value before the adjustment. By setting the transmit power as a function of a target value, more consistent contrast agent imaging may be provided despite the use of multiple foci or changes in the location of one of more foci or other imaging parameters. By automating setting the transmit power or as a function of the target value, user adjustment may be avoided and workflow will be improved. A target acoustic pressure, intensity or mechanical index value may be used for high power, more destructive imaging as well.

In a first aspect, a method is provided for controlling an intensity level in contrast agent imaging. A target value associated with an intensity of acoustic energy is determined. The target value corresponds to limited destruction of contrast agents. A transmit power is automatically set as a function of the target value with the processor. A substantially uniform intensity is maintained as a function of time, location or combinations thereof based on the setting of transmit power as a function of the determined target value.

In a second aspect, a system is provided for controlling an intensity level in contrast agent imaging. A control processor is operable to determine a target value associated with an intensity of acoustic energy in response to configuration of a system for contrast agent imaging with limited destruction of contrast agents. The control processor is also operable to automatically set a transmit power as a function of the target value. A transmit beamformer is operable to generate a transmit waveform as a function of the transmit power. [0011] In a third aspect, a method is provided for contrast agent imaging using two or more transmit focal depths. Different transmit amplitudes are set for different foci. The different foci are at different depths for a same image. A target acoustic pressure is substantially maintained at the different foci as a function of the different transmit amplitudes.

In a fourth aspect, a method is provided for contrast agent imaging with changes in imaging parameters during an imaging session. A transmit amplitude is set as a function of a first set of imaging parameter values. A first acoustic pressure at a first location is a function of the transmit amplitude and the imaging parameter values. At least one of the imaging parameter values is reset or changed during the imaging session. The transmit amplitude is reset or changed in response to the resetting of the imaging parameter value. The altered transmit amplitude is operable to provide a second acoustic pressure at the first location. A ratio of the second acoustic pressure to the first acoustic pressure is between 1.3 and 0.7.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a graphical representation of relationship of mechanical index at a focus, contrast agent persistence and signal-to-noise ratio in contrast agent imaging;

FIG. 2 is a graphical representation of differences in mechanical index between different focal depths for contrast agent imaging;

FIG. 3 is a block diagram of one embodiment of a system for controlling an intensity level in contrast agent imaging; and

FIG. 4 is a flow chart diagram of one embodiment of a method for controlling an intensity level in contrast agent imaging.

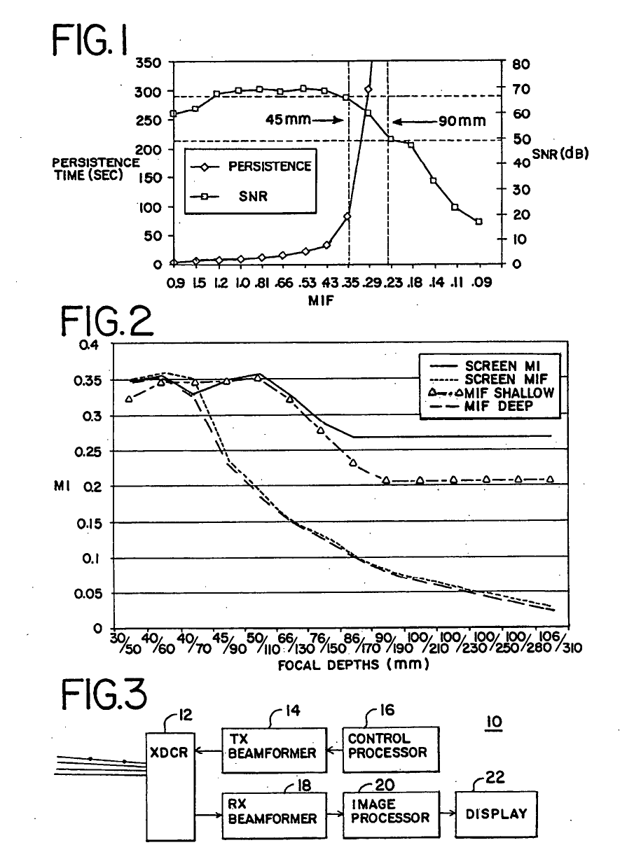

### DETAILED DESCRIPTION OF THE DRAWINGS AND PRESENTLY PREFERRED EMBODIMENTS

Transmit voltage, current, power or energy is used to maintain a targeted or uniform mechanical index, intensity or pressure. Any of various now known or later developed measures of the likelihood of contrast agent destruction, or lack of destruction, may be used. The mechanical index is a measure of intensity, energy, power, pressure, frequency or combinations thereof. Other measures, such as directly measuring any of the characteristics, may be used. For limiting contrast agent destruction, the target value is associated with a location within a scan plane, such as a location adjacent to the transducer, at a focal region or spaced away from the transducer and the focal region. By providing a uniform or similar mechanical index between two or more foci, near field contrast agent destruction is minimized while overall depth of penetration and sensitivity is improved. By maintaining a uniform or similar mechanical index in response to a change in imaging parameters, more uniform imaging is maintained throughout an imaging session.

The amplitude of a transmit waveform applied to a transducer is altered to control or limit the destruction of contrast agent. The amplitude of the waveform is adjusted by controlling a voltage, current or combinations thereof. Alterations of the transmit amplitude result in an increase or decrease in the transmit power or energy. After conversion to acoustic energy by the transducer, a transmit acoustical waveform is output by each element with a corresponding energy, power or amplitude level. Changes in the transmit level for electrical or acoustical waveforms are performed at each or some of the elements within a transmit aperture. By changing the transmit power at an element, a transmit power associated with the transmit aperture is also changed. The transmit power associated with an aperture may alternatively be changed by controlling the aperture, such as reducing a transmit power by reducing a number of elements within a transmit aperture. The mechanical index is linearly related to the peak negative pressure in a field. The peak negative pressure is a function of the peak voltage applied at the transducer element. By adjusting the voltage or amplitude, the mechanical index may be controlled without more complex changes to the imaging system. Additional changes may be provided.

FIG. 3 shows one embodiment of a system 10 for controlling an intensity level in contrast agent imaging. The system 10 includes a transducer 12, a transmit beamformer 14, a control processor 16, a receive beamformer 18, an image processor 20 and a display 22. Additional, different or fewer components may be provided, such providing a controller common to both the transmit and receive beamformers 14, 18 or a system without the image processor 20 or the display 22. The system 10 is a medical diagnostic ultrasound imaging system in one embodiment, but therapeutic or other ultrasound systems may be used.

The transmit beamformer 14 is a digital circuit, analog circuit, application-specific integrated circuit, memory, delays, amplifiers, filters, waveform generators, digital-to-analog converters, combinations thereof or other now known or later developed transmit beamformer components. The transmit beamformer 14 generates a plurality of electrical waveforms. One or more waveforms is generated for each element for a given transmit event. Where two or more waveforms are generated for a same transmit event for each element, the transmit waveforms are relatively apodized and delayed, and then combined. The apodized and delayed waveforms for each element are applied to the transducer 12. Relatively apodization controls the focus or other beam characteristic of the transmitted acoustic energy responsive to the transmit waveforms. Apodization is implemented by altering the amplitude of one transmit waveform relative to another transmit waveform. The amplitude used for a given element or combination of energy across multiple elements is also a function of a desired transmit power. By altering the transmit power, the destruction of contrast agents may be limited. Alteration of the transmit power may also allow for generating a desired intensity, such as a desired mechanical index, at one or more locations or at a same location at different times. The transmit power is adjusted by varying an amount of voltage or current amplification of the transmit waveforms. Alternatively, the waveform is generated with a desired or adjustable transmit power.

The transducer 12 is a one-dimensional, two-dimensional, multi-dimensional, 1.25D, 1.5D, 1.75D or other transducer array. The transducer 12 includes one or more elements of piezoelectric, capacitive membrane or other structures. The transducer 12 converts the electrical transmit waveforms into acoustical waveforms at each of the elements within a transmit aperture. The waveforms propagate to one or more focal regions. Acoustic echoes return to the transducer 12. The transducer 12 converts the acoustic echoes into received electrical signals at each of the elements of a receive aperture.

A plurality of transmit events are performed to transmit acoustic energy along different scan lines. A 1-, 2- or 3-dimensional region of interest is scanned. The mechanical index at any location within a scanned region is limited to 1.9. Other limitations may be used. To avoid contrast agent destruction, the mechanical index of 0.6, 0.5, 0.4 or other value or lower is desired for locations within the scan region. The acoustic energy generated by each element sums at each location to a different extent. Typically, the maximum mechanical index is at an electric or mechanical focus of the transducer. The strength of the reflected echoes is a function of the mechanical index. A greater amplitude echo signal is received from acoustic reflections associated with a higher mechanical index.

The receive beamformer 18 generates beamformed data for one or a plurality of scan lines. Beamformed data is formed by summing relatively delayed and apodized receive signals reflected from contrast agents, fluid or tissue. The image processor 20 is a detector and/or scan converter. The image processor 20 detects an intensity, power, velocity, energy, variance or other information. In one embodiment, the image processor 20 or the receive beamformer 18 is operable to combine data for isolating contrast agent information. For example, a plurality of pulses is transmitted to a same area or region but with different relative amplitudes. By combining the receive signals responsive to the different amplitude transmitted pulses, information at a cubic fundamental may be isolated. The cubic fundamental is responsive to contrast agents. As another example, two pulses are transmitted out of phase with each other. The responsive receive signals are then combined, isolating information at even harmonics, such as the second harmonic. Any now known or later developed contrast agent detection mode may be used, such as modes relying on loss of correlation, isolation of information at particular frequency bands, relative receive and/or transmit phasing, relative receive and/or transmit weighting, and/or identifying data associated with a particular range of motion. The image processor 20 outputs the image data to the display 22. The image is generated on the display 22 for viewing by a clinician.

The image or images displayed on the display 22 or the associated ultrasound data responsive to the receive signals is obtained as a function of the transmitted acoustic energy. Where different portions of the same image are responsive to varying mechanical indexes or other intensities, the resulting image may have undesired variation. Similarly, where different images within a sequence of image are responsive to different mechanical indexes due to changes of imaging parameters, the resulting images may be less desirable. The control processor 16 controls the transmit power of the transmit beamformer 14 to provide a more uniform intensity as a function of location, time or combinations thereof.

The control processor 16 is a general processor, digital signal processor, application-specific integrated circuit, analog circuit, digital circuit, beamformer controller, system controller, combinations thereof or other now known or later developed device for obtaining a target value and/or setting a transmit power of the transmit beamformer 14. The control processor 16 implements the power management control algorithm. Any now known or later developed calculations, considerations or other aspects of power management may be included as well as the maintenance of a substantially uniform intensity.

The control processor 16 is operable to determine a target value associated with an intensity of acoustic energy. For example, the control processor determines a target value as a particular mechanical index or other representation of acoustic pressure in response to configuration of a system for contrast agent imaging with limited destruction of contrast agents. Target value is determined automatically in response to the configuration or other settings, such as selecting a 0.4 or other MI value as a target value in response to a user selection of a contrast agent imaging mode of operation.

The control processor 16 is also operable to set the transmit power of the transmit beamformer 14 as a function of the target value. In response to configuring the system 10 for imaging contrast agents with limited destruction, the transmit power is set to avoid exceeding the target value or to avoid exceeding the target value by a tolerance amount. The control processor 16 implements an algorithm for determining the transmit power that is based on the target value. Rather than starting with a maximum possible transmit power and arbitrarily assigning a reduction in power based on a manual user setting, the algorithm identifies a transmit power in order to obtain the target or similar value of acoustic pressure or an intensity.

By maintaining a substantially uniform intensity as a function of time, location or combinations thereof based on setting the transmit powers as a function of the target value, the control processor 16 may provide more uniform contrast agent imaging. In one embodiment, a substantially uniform intensity is provided where the intensities at a same location as a function of time or different foci locations in a same image are within 0.05 of each other or of the mechanical index target value 0.2, 0.1 or other ranges of tolerance to provide a substantially uniform intensity may be used. The algorithm seeks to obtain a same or target value intensity. For a deep focus or foci, a desired mechanical index or other intensity may not be achieved due to a mechanical focus of a transducer and greater tissue or contrast attenuation at the deeper foci. A shallow focus or foci are maintained at the target value, but the deeper focus or foci may have a reduced intensity to avoid exceeding a target value or a tolerance of the target value at the mechanical focus or elsewhere along a scan line.

The control processor 16 maintains the substantially uniform intensity in any of various situations. For example, control processor 16 is operable to automatically set the transmit power for deeper and shallower foci in a same image and along a same or adjacent scan lines. The transmit powers for the different foci are set as a function of the target value. Substantially uniform intensity is then provided at each of the foci. As another example, the control processor 16 is operable to automatically reset or alter from one transmit power to a different transmit power in response to alteration of an imaging parameter during an imaging session. Various imaging parameters may affect the acoustic intensity or pressure at a given location. Rather than maintaining a same transmit power after alteration of an imaging parameter, the transmit power is adjusted to maintain a substantially uniform intensity as a function of time from before the alteration to after the alteration.

Ultrasound images are typically acquired during an imaging session. Imaging sessions may last from minutes to hours. Typically, an imaging session is associated with one or a few time periods of continuous acquisition of images. For example, in contrast agent imaging, an imaging session corresponds to the ultrasound examination of a patient before, during and/or after a single injection of contrast agents. The target intensity is maintained during a period of continuous imaging or an imaging session.

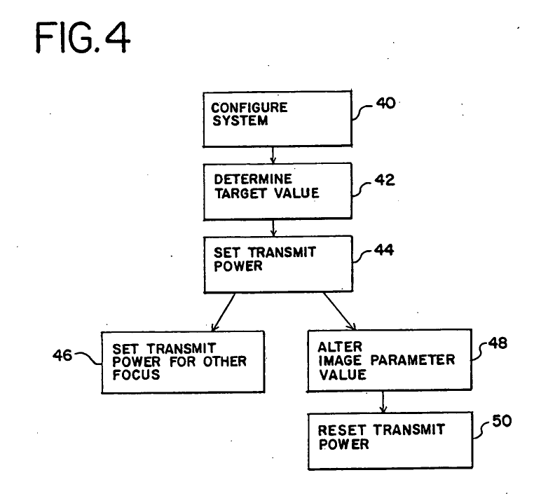

FIG. 4 shows one embodiment of a method for controlling an intensity level in contrast agent imaging. The acts of the method shown are for contrast agent imaging using two or more transmit focal depths or for contrast agent imaging with changes in imaging parameters during an imaging session. Controlling the intensity level for contrast agent imaging as a function of both time and location may be used in other embodiments. Additional, different or fewer acts may be provided. For example, the target value is determined in act 42 without the configuration of a system in act 40. As another example, act 46 for setting a transmit power for an additional focal point is not provided, or acts 48 or 50 for changing transmit power as a function of an altered imaging parameter are not provided. As yet another example, act 44 is provided without the determination of a target value in act 42. The method of FIG. 4 is implemented using a system described above for FIG. 3 or a different system.

In act 40, an ultrasound system is configured for imaging contrast agents with limited destruction of the contrast agents. For example, the user selects a contrast agent imaging application or a particular detection mode. The selection corresponds to an automatic arrangement or establishment of one or more values for generating an image. For example, a detection mode, filters, scan format, waveforms, scan line density, frequency, focal depth, apodization, aperture or other imaging parameter value is set as a function of a selected configuration. Other factors may be included in setting the parameter values, such as a type of ultrasound transducer. As yet another example of configuration of an ultrasound system, the user manually selects one or more values for different imaging parameters rather than selecting an application. The user may adjust or alter values. For example, the user changes a transmit power by adjusting a knob or other user control or a user input. The change of an imaging parameter, selection of an application or establishing another configuration invokes the algorithm for providing a substantially uniform acoustic intensity as a function of time, location or combinations thereof. The algorithm determines the target value and sets the transmit power automatically. The algorithm is invoked automatically in response to configuring the ultrasound system.

In act 42, a target value is determined. The target value corresponds to a particular location, such as at one or more focal locations. Alternatively, the target value is associated with a location spaced away from the focus. Target value may be applied as a function of time for a given location or as a function of multiple locations, such as for different focal depths used in a same image. The target value is associated with an intensity of acoustic energy. For example, a target value is a mechanical index, acoustic pressure, intensity, power, energy or other type of value. In one embodiment, the target value is a transmit power associated with an entire aperture. The target value corresponds to limiting destruction of contrast agents in one embodiment, such as a mechanical index of 0.6 or lower.

The target value is determined in response to user input. For example, the user defines a target mechanical index at a focal point or at foci at the beginning of an examination or during an examination. A control knob, slider, voice recognition, or other user input device is manipulated by the user to indicate a desired target value. The target value is directly selected or determined from a user setting of a transmit power. In one embodiment, the target value is set as a mechanical index or other value based on user input, such as performed by manual adjustment of a power control setting the transmit powers in act 44. In an alternative embodiment, the target value is selected from a look-up table. Any of various factors may be considered for selecting the target value, such as the transducer used, the depth of imaging, the transmit frequency, the type of detection (e.g. low mechanical index contrast agent imaging as opposed to contrast agent destructive imaging) or other factors. In yet another alternative embodiment, the algorithm disclosed in U.S. Pat. No. ______ (application Ser. No. 10/077,499), the disclosure of which is incorporated herein by reference, or other automatic method for determining mechanical index or other target value is used. The algorithm is sensitive to contrast agent destruction to determine a mechanical index or other acoustic intensity associated with limiting contrast agent destruction.

In act 44, the transmit power is set as a function of the target value. The transmit power is automatically set with the control processor. By implementing an algorithm to provide the highest voltage for the transmit beamformer that results in an intensity at or within the tolerance of the target value allows contrast agent imaging with more consistent acoustic pressures. The transmit voltage for each element of an array or transmit aperture is set to have the desired transmit power for the element or for the array. If the target value is a mechanical index value, the transmit voltage is set to provide the desired mechanical index value. By setting the transmit voltages or currents for each of the elements, a transmit power for the entire array or transmit aperture associated with obtaining the desired target value at the focal region or other location is provided. Since the transmit power is set automatically with the processor, further subjective user or manual input is avoided. Alternatively, some manual adjustment or manual setting of the transmit power is provided.

Aperture, apodization, tissue attenuation or system beamforming are modeled or experimentally determined to provide a relationship between desired acoustic pressure and a transmit amplitude. The relationships may be adjusted further by input from a user or initially established in response to user input.

In act 44, the transmit power or associated transmit amplitudes are set as a function of a set of imaging parameter values. The acoustic pressure at a desired location is a function of the transmit amplitude and the imaging parameter values. After the imaging parameter values are selected, the transmit amplitude is adjusted or set to provide the desired target acoustic pressure. The transmit amplitude is set to provide acoustic pressure within a threshold of the target, such as providing a ratio of the acoustic pressure to the target acoustic pressure between 1.3 and 0.7 or other range of values.

In act 46, the transmit power for an additional spatial location, such as an additional focal location, is provided. The transmit powers and associated voltages for the two different focal depths are set as a function of the same target value. The transmit powers for each of the foci may be different. For example, the transmit amplitudes for a shallower focus are less than the transmit amplitudes for a deeper focal region. In a low mechanical index contrast agent image, the transmit amplitude of each element of an aperture for one focal depth is set as a function of a target acoustic pressure. The transmit amplitude for each element in another transmit aperture for a different focal depth is also set as a function of the target acoustic pressure. The transmit amplitude for at least one element in common to both of the transmit apertures is different. To maintain the target value, such as a target mechanical index, the relative weighting of the voltages applied for two different foci and the resulting transmit power are varied.

Since the mechanical index is linearly related to the voltage, the voltage associated with a higher mechanical index focal position is reduced by the ratio of the target mechanical index divided by the original or target mechanical index. Other calculated or experimentally determined linear or non-linear functions relating intensity to waveform amplitude may be used. Alternatively, the voltage associated with one focal position is increased and the voltages associated with another focal position are decreased. In yet another embodiment, voltage associated with a lesser mechanical index focal position is increased to provide a mechanical index substantially similar to a mechanical index at a different focal position. Before, during or after setting the transmit voltages, the target value may be increased or decreased to provide an overall increase or decrease in total output power.

In one embodiment, a single transmit firing along a same scan line or adjacent scan lines has multiple foci. Through relative delays and apodization, more than one focal point along a scan line is provided for a single transmission. The single transmission may correspond to transmitting two or more sets of waveforms combined together. Each set of waveforms is independently apodized and delayed to provide the different focal positions. For example, dynamic transmit focusing is provided where waveforms associated with each unique focal position or transmit delay profile have separately controlled voltages, currents or amplitudes to provide the desired target value at each focal point or other location. Alternatively, a single transmit waveform is generated for each element with relative delays and apodization to provide a dual foci, three or more foci, or a continuous focus. Transmit voltage, current or amplitude for each transmit waveform applied to an element is set to provide a uniform or substantially uniform acoustic intensity at two or more of the focal positions.

In an alternative embodiment, sequential transmissions of acoustic energy are performed to the different focal depths. The transmit voltage, current or amplitude of each of the waveforms applied to the transducer elements for generating an acoustic beam with the desired target value is set separately for each of the sequential transmissions. The desired transmit voltage for each waveform is calculated for each focus separately.

For low mechanical index contrast agent imaging, transmit voltages less than a maximum power are available. The available overhead of transmit power may allow adjustment of waveforms for one or more focal positions to match a target value. Transmit voltages for one, some or all of the focal positions may be decreased, increased or maintained. Since the transmit power is automatically set, the user may be provided with a substantially uniform image without further input or manual adjustment. In one embodiment, the transmit powers for different focal positions are set automatically before imaging is begun. As an alternative to setting the foci to a target value, the foci are set to be within a particular threshold of intensity with each other, defining a target value. The target value is then stored for later use. Where a deeper or deepest focal point is beyond the elevational focus to the extent that intensity at the elevational or mechanical focus of the transducer is greater than the intensity at the deeper focal point, the target value is applied to the mechanically focused location or other location spaced away from the deepest focal location. In the embodiment where the intensity is higher at the mechanical focus than a deeper focal location, the target value is set based on the intensity at one or more near field focal points or the elevational focal point.

A substantially uniform intensity is maintained as a function of location. The substantially uniform intensity is based on setting a transmit power as a function of the determined target value. For example, intensity is maintained as substantially uniform if the mechanical index is within 0.1 or a target mechanical index value or 0.05. Other values may be provided. A ratio of the acoustic pressure at each of the foci is between 1.2 and 0.8, 1.1 and 0.9, 1.05 and 0.95, or other range of symmetrical or asymmetrical values about equal pressures. Where two or more focal depths are used for a same image, the intensity is maintained and is substantially uniform for a plurality of the different focal depths along a same or adjacent scan lines. The target acoustic pressure is maintained at the different foci based on using different transmit amplitudes for the different foci. By reducing or increasing a transmit amplitude to provide an acoustic pressure substantially equal to the target acoustic pressure, a target acoustic pressure is substantially maintained and the image or focal positions are more likely uniformly responsive to contrast agents.

In act 48, an imaging parameter is altered during an imaging session. The sequence or plurality of images within an imaging session is responsive to the various imaging parameters. By altering an imaging parameter, the intensity at a focal point or other location may be altered. For example, the user alters a transmit frequency, aperture size, number of elements in a transmit aperture, aperture position, apodization, transmit focus location, number of transmit events per image, number of unique transmit lines in an image, scan line density, imaging mode or combinations thereof. The alteration may change the acoustic pressure without resetting a transmit amplitude.

To provide a substantially uniform intensity during the imaging session, transmit power is reset or altered in act 50. The uniform intensity is substantially maintained as a function of time by altering or resetting the transmit power. For example, substantially uniform intensity is maintained by providing a mechanical index within a scan region that is within 0.05, 0.1, 0.2 or other value of a target mechanical index or mechanical index of a previous or subsequent image. A ratio of the acoustic pressures at a same location in different images associated with different imaging parameters are between 1.3 and 0.7, 1.2 and 0.8, 1.1 and 0.9, 1.05 and 0.95 or other range of variants. The target mechanical index and mechanical index measure is a maximum for the spatial locations of an image, so other locations may have an intensity less than the maximum value. The mechanical index at a particular location is maintained to provide a substantially uniform intensity throughout an imaging session.

The transmit power is reset from an original transmit power to a different transmit power in response to the altered imaging parameter. By automatically performing the alteration, a substantially uniform intensity is provided despite the change in the imaging parameter without further user input. By adjusting the transmit power to provide a substantially uniform intensity at a focal location or other location following a parameter change, more consistent contrast agent imaging is provided. The mean, maximum or other measure of the intensity of the different images associated with different imaging parameters may be different than an overall or initially established target mechanical index. Further adjustment of the transmit voltages may be automatically performed to provide an overall transmit power to achieve the target intensity or other value, such as associated with avoiding or limiting destruction of contrast agent.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I claim:

1. A method for controlling an intensity level in contrast agent imaging, the method comprising:
(a) determining a target value associated with an intensity of acoustic energy, the target value corresponding to limiting destruction of contrast agents;
(b) automatically setting a first transmit power as a function of the target value with a processor; and
(c) maintaining a substantially uniform intensity as a function of time, location or combinations thereof based on setting the transmit power as a function of the determined target value.

2. The method of claim 1 wherein (a) comprises determining the target value for each of at least first and second different focal depths, wherein (b) comprises setting the first and a second transmit power for the first and second focal depths, respectively, as a function of the target value, the first transmit power different than the second transmit power, and wherein (c) comprises maintaining the substantially uniform intensity as a function of the first and second focal depths used for a same image.

3. The method of claim 1 further comprising:
(d) altering an imaging parameter during an imaging session, the images of the imaging session responsive to the substantially uniform intensity;
wherein (c) comprises re-setting from the first transmit power to a second transmit power in response to the altered imaging parameter, the second transmit power operable to maintain the substantially uniform intensity as a function of time from before the alteration of (d) to after the alteration of (d), wherein (c) is performed automatically in response to the alteration.

4. The method of claim 3 wherein (d) comprises altering transmit frequency, aperture size, number of elements, apodization, transmit focus location, number of transmit foci, number of transmit events per image, number of unique transmit lines in an image, an imaging mode or combinations thereof.

5. The method of claim 1 wherein (a) comprises determining a target mechanical index value at a focal location, and wherein (b) comprises setting a transmit voltage as a function of the target mechanical index value.

6. The method of claim 5 wherein (c) comprises maintaining the substantially uniform intensity to within 0.10 of the target mechanical index value.

7. The method of claim 5 wherein (c) comprises maintaining the substantially uniform intensity to within 0.05 of the target mechanical index value.

8. The method of claim 1 further comprising:
(d) configuring an ultrasound system for imaging contrast agents with limited destruction of the contrast agents;
wherein (a) and (b) are performed automatically in response to the configuration of (d).

9. The method of claim 1 wherein (c) comprises maintaining the substantially uniform intensity for a plurality of different focal depths along a same scan line for a same image.

10. The method of claim 1 wherein (c) comprises maintaining the substantially uniform intensity for a same location throughout an imaging session.

11. The method of claim 1 wherein (b) comprises setting the first transmit voltage for a first element of an array;
further comprising:
(d) automatically setting transmit voltages for other elements than the first element as a function of the target value with a processor.

12. A system for controlling an intensity level in contrast agent imaging, the system comprising:
a control processor operable to determine a target value associated with an intensity of acoustic energy in response to configuration of the system for contrast agent imaging with limiting destruction of contrast agents, the control processor operable to automatically set a first transmit power as a function of the target value; and
a transmit beamformer operable to generate a transmit waveform as a function of the first transmit power.

13. The system of claim 12 wherein the control processor is operable to maintain a substantially uniform intensity as a function of time, location or combinations thereof based on setting the first and a second transmit power as a function of the determined target value.

14. The system of claim 12 wherein the control processor is operable to automatically set the first transmit power for a first focal region and set a second transmit power for a second focal region deeper than the first focal region, both the first and second transmit powers set as a function of the target value such that a substantially uniform intensity is provided at the first and second focal regions for a same image.

15. The system of claim 12 wherein the control processor is operable to automatically reset from the first transmit power to a second transmit power in response to alteration of an imaging parameter during an imaging session, the second transmit power operable to maintain a substantially uniform intensity as a function of time from before the alteration to after the alteration.

16. The system of claim 13 wherein the control processor is operable to maintain the substantially uniform intensity to within 0.05 of a mechanical index target value.

17. The system of claim 12 wherein the control processor is operable to automatically determine the target value and set the first transmit power in response to configuration of the system for imaging contrast agents with limited destruction of the contrast agents.

18. A method for contrast agent imaging using two or more transmit focal depths, the method comprising:
(a) setting different transmit amplitudes for different foci, the different foci being at different depths for a same image; and
(b) substantially maintaining a target acoustic pressure at the different foci as a function of the different transmit amplitudes, respectively.

19. The method of claim 18 wherein (a) comprises:
(a1) setting a first transmit amplitude of each element of a first transmit aperture for a first focal depth as a function of a target acoustic pressure; and
(a2) setting a second transmit amplitude of each element of a second transmit aperture for a second focal depth as a function of the target acoustic pressure, the first focal depth different than the second focal depth and the first transmit amplitude different than the second transmit amplitude for at least one element common to both the first and second transmit apertures;
wherein the image is responsive to the settings of (a1) and (a2); and
wherein a ratio of a first acoustic pressure responsive to the first transmit amplitudes and a second acoustic pressure responsive to the second transmit amplitudes is less than 1.2 and greater than 0.8.

20. The method of claim 19 wherein the ratio is between 1.1 and 0.9.

21. The method of claim 18 wherein the image is a low mechanical index image corresponding to limited destruction of contrast agents.

22. The method of claim 18 wherein (a) and (b) comprise reducing a first transmit amplitude such that a first acoustic pressure at a first focal depth is substantially equal to the target acoustic pressure.

23. The method of claim 18 wherein (a) and (b) comprise increasing a first transmit amplitude such that a first acoustic pressure at a first focal depth is substantially equal to the target acoustic pressure.

24. The method of claim 18 further comprising sequentially:
(c) transmitting acoustic energy focused at a first focal depth of the different foci; and
(d) transmitting acoustic energy focused at a second focal depth of the different foci.

25. A method for contrast agent imaging with changes in imaging parameters during an imaging session, the method comprising:
(a) setting a transmit amplitude as a function of a first set of imaging parameter values, a first acoustic pressure at a first location being a function of the transmit amplitude and the imaging parameter values;
(b) resetting at least one of the imaging parameter values during the imaging session; and
(c) resetting the transmit amplitude in response to the resetting of the at least one of the imaging parameter values, the reset transmit amplitude operable to provide a second acoustic pressure at the first location, a ratio of the second acoustic pressure to the first acoustic pressure being between 1.3 and 0.7.

26. The method of claim 25 wherein the ratio is between 1.2 and 0.8.

27. The method of claim 25 wherein the ratio is between 1.1 and 0.9.

28. The method of claim 25 wherein the ratio is between 1.05 and 0.95.

29. The method of claim 25 wherein (a) comprises setting the transmit amplitude as a function of a target acoustic pressure, a ratio of the first acoustic pressure to the target acoustic pressure being between 1.3 and 0.7.

30. The method of claim 29 further comprising:
(d) configuring an ultrasound system for contrast agent imaging associated with limited destruction of contrast agents;
wherein (a) is performed automatically in response to the configuration of (d).

31. The method of claim 25 wherein (b) comprises altering transmit frequency, aperture size, number of elements, apodization, transmit focus location, number of transmit foci, number of transmit events per image, number of unique transmit lines in an image, an imaging mode or combinations thereof, the alteration operable to change the first acoustic pressure without the resetting of the transmit amplitude in (c).
